(19) United States
(12) Patent Application Publication
(10) Pub. No.: US 2020/0246454 A1
(43) Pub. Date: Aug. 6, 2020

(54) METHODS AND THERAPEUTIC COMPOSITIONS FOR TREATMENT OF ALLERGY

(71) Applicant: ReAlign Therapeutics, Inc., Hidden Hills, CA (US)

(72) Inventors: Vasco Santos SINGH, Hidden Hills, CA (US); Akhil Ram GUTTA, Thousand Oaks, CA (US); Arjun J. GUTTA, Thousand Oaks, CA (US); Samarth Eshwar KADABA, Newbury Park, CA (US)

(73) Assignee: ReAlign Therapeutics, Inc, Hidden Hills, CA (US)

(21) Appl. No.: 16/781,892

(22) Filed: Feb. 4, 2020

Related U.S. Application Data
(60) Provisional application No. 62/801,003, filed on Feb. 4, 2019.

(51) Int. Cl. A61K 39/35; A61K 35/747; A61K 35/745; A61K 35/742; A61K 47/36; A61K 47/26; A61K 31/593

(52) U.S. Cl. CPC A61K 39/35; A61K 35/747; A61K 35/745; A61K 31/593; A61K 47/36; A61K 47/26; A61K 35/742

(57) ABSTRACT

Disclosed are methods, and compositions of matter useful for preventing/treating allergies. More specifically, methods of treating a subject who has developed allergies after and/or as a result of taking antibiotics that disrupt the gut microflora are provided herein. Specifically, methods herein involve treating an allergy sufferer through the administration of one or more probiotics, one or more allergens, with or without one or more prebiotics.

### CROSS-REFERENCE TO RELATED APPLICATIONS

This application claims priority to U.S. Provisional Application No. 62/801,003, filed Feb. 4, 2019, which is incorporated by reference in its entirety.

### FIELD OF THE INVENTION

The teachings herein are directed to compositions and methods for treating allergies in subjects, such as subjects who are suffering from allergies after taking antibiotics.

### BACKGROUND

Allergic states affect humans and companion animals and some cases allergic reactions are threatening to life. Allergic reactions result when a subject's immune system responds to an allergen. Typically, there is no allergic reaction the first time a subject is exposed to a particular allergen. However, it is the initial response to an allergen that primes the system for subsequent allergic reactions. In particular, the allergen is taken up by antigen presenting cells which endocytose/phagocytose said allergen and then display allergen fragments to T-cells in an MHC restricted manner [1, 2].

Food allergy symptoms may affect the skin, nasal and oral mucosa, conjunctivae, gastrointestinal tract or, in severe cases, the respiratory tract and cardiovascular organs. Immunoglobulin E (IgE)-mediated symptoms appear rapidly after exposure to the offending allergen, whereas non-IgE-mediated symptoms are typically delayed [3]. T-cells, in particular CD4+ “helper” T-cells, respond by secreting a collection of cytokines that have effects on other immune system cells. The profile of cytokines secreted by responding CD4+ T-cells determines whether subsequent exposures to the allergen will induce allergic reactions. Two classes of CD4+ T-cells (Th1 and Th2; T-lymphocyte helper type) influence the type of immune response that is mounted against an allergen. Exposure of CD4+ T-cells to allergens can also activate the cells to develop into Th2 cells, which secrete IL-4, IL-5, IL-10, and IL-13. IL-4 production stimulates maturation of B cells that produce IgE antibodies specific for the allergen. These allergen-specific IgE antibodies attach mast cell and basophil receptors, where they initiate a rapid immune response to the next exposure to allergen. When the subject encounters the allergen a second time, the allergen is quickly bound by these surface-associated IgE molecules, resulting in the release of histamines and other substances that trigger allergic reactions. Subjects with high levels of IgE antibodies are known to be particularly prone to allergies.

Food allergies are known to be highly prevalent. One report suggests that 6% of adults and up to 8% of children are affected [4]. Unfortunately, it is believed that the prevalence of allergy and anaphylaxis to food based allergens is increasing [5-7]. Standard means of controlling/living with allergies are following strict food avoidance and carrying an epinephrine auto-injector (EAI) in the event of a reaction [8]. Despite efforts at avoidance, severe reactions may occur in up to a third of food allergic children.5 Given risk for accidental reactions and persistence of FA beyond childhood, there is an unmet need for therapies for FA. A number of allergen-specific methods are being studied, and may become commercially available in the coming years. In this review, we will discuss food allergen-specific immunotherapies (AITs) that are being evaluated in humans.

At present, various interventions to de-sensitize patients have been explored including low dose intravenous administration of the allergen, oral tolerance, and administration of modified form of allergens. Unfortunately, the present state of the art lacks a consistent, reproducible and safe manner of effectively inducing immunological tolerance to allergens.

Current treatments for allergies generally involve treatment of symptoms or prevention. For example, avoidance of allergens is commonly thought. Unfortunately, this is not very effective with many times allergens not being noticed. Other treatments include epinephrine to treat the many signs of allergy. This compound acts on a number of receptors in the body to exert its effects. First, it causes constriction, or tightening, of the blood vessels, which decreases swelling and also helps to increase blood pressure. It also increases the heart's contraction and heart rate, which can help to prevent or reverse cardiovascular collapse. Epinephrine relaxes the muscles around the airways in the lungs, helping the airways to open up. Finally, it prevents the release of additional allergic chemicals, which aids in stopping further progression of the reaction.

Patients with allergies sometimes develop them because of living in an ultraclean environment. When one is not exposed to a diverse set of bacteria, the microbiome does not form properly and as a result normal tolerogenic processes do not have an opportunity to become established.

The invention provides means of inducing tolerogenesis and/or reducing allergic reactions.

### SUMMARY

Certain embodiments are directed to a method for inducing/augmenting tolerance, and/or treating, preventing and/or reducing the risk and/or symptoms of allergy in a subject, comprising: identifying a subject suffering from an allergy; administering to the subject a composition comprising of; a) one or more probiotics; b) one or more allergens; and optionally; c) one or more prebiotics.

In certain aspects, said tolerance is associated with augmentation of numbers and/or activity of T regulatory cells.

In certain aspects, said oral tolerance is associated with augmentation of numbers and/or activity of B regulatory cells.

In certain aspects, said oral tolerance is associated with a reduction in allergy-causing potential of an allergen.

In certain aspects, said probiotic is one or more probiotics selected from a group comprising of: a) Clostridium Butyricum; b) Lactobacillus Rhamnosus and; c) Bifidobacterium Longum.

In certain aspects, said prebiotics are one or more prebiotics selected from a group comprising of; a) inulin; b) vitamin D3; and c) oligosaccharides.

In certain aspects, said allergens are derived from extracts, purified or non-purified from one or more sources of allergy evoking materials selected from a group comprising of: a) peanuts; b) wheat; c) soy; d) fish; e) shellfish; f) tree nuts; and g) milk.

In certain aspects, said tolerance is oral tolerance.

In certain aspects, said tolerance is sublingual tolerance.

In certain aspects, said tolerance is intravenous tolerance.

In certain aspects, said tolerance is epicutaneous tolerance.

In certain aspects, said tolerance is low dose tolerance.

In certain aspects, said allergy is high dose tolerance.

In certain aspects, said allergy is naturally occurring.

In certain aspects, said allergy occurs as result of a microbiome dysbiosis.

In certain aspects, said microbiome dysbiosis occurs in the gut microbiome.

In certain aspects, said microbiome dysbiosis occurs as result of exposure to one or more antibiotics.

In certain aspects, said allergy occurs as result of exposure to one or more antibiotics.

In certain aspects, said allergen is a food allergen.

In certain aspects, said allergen is a peanut allergen.

In certain aspects, said peanut allergen is selected from the groups consisting of Ara h 1, Ara h 2, and Ara h 3.

In certain aspects, said food allergen is a milk allergen.

vsaid food allergen is a shellfish allergen.

In certain aspects, the allergen is an environmental allergen.

In certain aspects, said environmental allergen is grass pollen.

In certain aspects, wherein said environmental allergen is tree pollen.

In certain aspects, said allergen is a drug.

In certain aspects, said allergen is a pollen.

In certain aspects, said allergen is an insect venom antigen.

In certain aspects, said allergen is an antigen possessing a majority of linear epitopes.

In certain aspects, the allergen is a peptide.

In certain aspects, the allergen is a collection of peptides.

In certain aspects, the allergen as admixed with and an adjuvant.

In certain aspects, said adjuvant possess at least one or more cytokines.

In certain aspects, said adjuvant possesses ability to induce immunological effects capable of suppressing production of Th2 cytokines.

In certain aspects, said Th2 cytokines are selected from a group comprising of: a) IL-4; b) IL-5; c) IL-13; d) IL-10; and e) IL-35.

In certain aspects, said adjuvant possesses ability to induce immunological effects capable of augmenting Th1 cytokines.

In certain aspects, said Th1 cytokines are selected from a group comprising of: a) IL-2; b) IL-7; c) IL-12; d) IL-15; e) IL-18; f) interferon gamma; and g) TNF-alpha.

In certain aspects, the step of administering the probiotic is performed within one month of the step of administering the allergen.

In certain aspects, the step of administering the probiotic is performed within one week of the step of administering the allergen.

In certain aspects, the step of administering the probiotic is performed within 48 hours of the step of administering the allergen.

In certain aspects, the step of administering the probiotic is performed within 24 hours of the step of administering the allergen.

In certain aspects, the step of administering the probiotic is performed within 8 hours of the step of administering the allergen.

In certain aspects, the step of administering the probiotic is performed within 2 hours of the step of administering the allergen.

In certain aspects, the step of administering the probiotic is performed within 1 hours of the step of administering the allergen.

In certain aspects, the step of administering the probiotic is performed within 30 minutes of the step of administering the allergen.

In certain aspects, said allergen is modified in a manner to decrease allergic potential while retaining or possessing augmented tolerogenic potential.

In certain aspects, said modification comprises alteration of at least one cysteine residue so as to remove at least one disulfide bond from the structure of said allergen.

In certain aspects, said modified allergen is utilized if decreased affinity to IgE specific for said allergen is observed.

In certain aspects, said decreased affinity to IgE specific for said allergen implies lower affinity as compared to the non-modified allergen.

In certain aspects, said modification comprises a reducing step and a step of treating with a cross-linking agent.

In certain aspects, said cross-linking agent is an aldehyde.

In certain aspects, the methods further comprise an alkylating step.

In certain aspects, the step of treating with a cross-linking agent is carried out after the reducing step.

In certain aspects, the reducing step is carried out prior to the alkylating step.

In certain aspects, the reducing step is carried out using a reducing agent selected from the group consisting of 2-mercaptoethanol (.beta.-ME), dithiothreitol (DTT), dithioerythritol, cysteine, homocystein, tributylphosphine, sulfite, tris(2-carboxyethyl) phosphine (TCEP), sodium (cyano) borohydride, lye, glutathione, E-mercapto ethylamine, thioglycollic acid, methyl sulfide, ethyl sulfide and combinations thereof.

In certain aspects, said alkylating step is carried out using an alkylating agent selected from the group consisting of N-ethylmalimide, cystamine, iodoacetamide, iodoacetic acid, alkylhalogenides; alkylsulfates; alkenes, enzymes, and combinations thereof.

In certain aspects, the allergen is a protein comprising cysteine residues.

In certain aspects, said composition is capable of reducing Th2 immunity in a mammal.

In certain aspects, said Th2 immunity comprises enhanced production of one or more cytokines selected from a group consisting of: a) IL-4; b) IL-5; c) IL-13; d) IL-10; and e) IL-35.

In certain aspects, said composition is capable of enhancing Th1 immunity in a mammal.

In certain aspects, said Th1 immunity comprises enhanced production of cytokines selected from a group consisting of: a) IL-2; b) IL-7; c) IL-12; d) IL-15; e) IL-18; f) interferon gamma; and g) TNF-alpha.

In certain aspects, Th2 immune is capable of producing enhanced allergic response.

In certain aspects, said enhanced allergic response comprises upregulated production of histamine.

In certain aspects, said enhanced allergic response comprises increased production of IgE.

In certain aspects, said Th2 response is associated with enhanced activity of STAT6.

In certain aspects, said Th2 response is associated with enhanced activity of GATA3.

In certain aspects, said Th1 response is associated with enhanced activity of T-BET.

In certain aspects, said Th1 response is associated with enhanced activity of STAT4.

In certain aspects, said prebiotic/probiotic composition enhances gut barrier function.

In certain aspects, said gut permeability reduction is associated with suppression of bacterial translocation.

In certain aspects, said prebiotics are selected from a group comprising of: inulin; vitamin D3; oligosaccharides, various gums (guar gum, xanthan gum, locust been gum), carob seed flour, oat bran, rice bran, barley, modified or unmodified starch and suitable partial hydrolysates thereof, partially hydrolysed inulin, natural or synthetic/biosynthetic oligofructoses, fructo-oligosaccharides (FOS), lactulose, galactomannan and suitable hydrolysates thereof, indigestible polydextrose, indigestible dextrin and partial hydrolysates thereof, trans-galacto-oligosaccharides (GOS), xylo-oligosaccharides (XOS), acemannan, lentinan or beta-glucan and partial hydrolysates thereof, polysaccharides P and K (PSP, PSK), tagatose, various fungal oligosaccharides and polysaccharides,

In certain aspects, said oral tolerance is associated with reduction of symptoms in allergy patients.

In certain aspects, said oral tolerance is associated with modulation of antigen presenting cell function.

In certain aspects, said modulation of antigen presenting cell function involves decreasing ability to stimulating Th2 immunity.

In certain aspects, said composition increases production of butyrate.

In certain aspects, methods of ameliorating one or more symptoms of an allergy in a subject are provided, comprising:
a) identifying a subject suffering from an allergy;
b) administering a probiotic to the subject; and
c) exposing the subject to an allergen that is causative of said allergy, in an amount sufficient to illicit an immune response, but not an anaphylactic response.

In certain aspects, said probiotic is selected from the group consisting of: a) Clostridium Butyricum; b) Lactobacillus Rhamnosus and; c) Bifidobacterium Longum.

In certain aspects, subject is administered a prebiotic before administration of the probiotic.

In certain aspects, said prebiotic is selected from the group consisting of: a) inulin; b) vitamin D3; and c) an oligosaccharide.

In certain aspects, said allergen is derived from a source selected from the group consisting of: a) peanuts; b) wheat; c) soy; d) fish; e) shellfish; f) tree nuts; g) dairy, and h) eggs.

In certain aspects, said allergen is derived from a source selected from the group consisting of: a) animal dander, b) pollen, c) grass, d) mold, e) insect stings, f) dust mites, g) latex, and h) animal saliva.

In certain aspects, said allergy occurs after or as a result of microbiome dysbiosis in the gut.

In certain aspects, said allergy occurs after or as a result of exposure to one or more antibiotics.

In certain aspects, exposing said allergen comprises internally administering the allergen to the subject.

In certain aspects, exposing said allergen comprises external exposure of the allergen to the subject.

In certain aspects, the probiotic and the allergen are administered contemporaneously or within 2 hours of each other.

In certain aspects, the subject is administered a probiotic comprising: Lactobacillus Rhamnosus and Bifidobacterium Longum.

In certain aspects, the probiotics are administered at between 1×10⁸ and 1×10¹² CFUs.

In certain aspects, the subject is further administered Bifidobacterium infantis.

In certain aspects, the subject is further administered Bifidobacterium Acidophilus.

In certain aspects, the probiotics are administered at least 7 days.

In certain aspects, exposing the allergen to the subject comprises an allergen immunotherapy protocol

### DESCRIPTION OF THE INVENTION

The invention teaches the previously unrecognized utilization of probiotics, as well as prebiotics and probiotics in order to augment the process of tolerance induction. In the practice of the invention, allergens may be administered together with said prebiotics, probiotics, or prebiotics and probiotic formulations in a frequency and concentration sufficient to induce tolerance and/or augment the process of tolerogenesis. Various types of tolerogenesis are envisioned in the current invention including oral tolerance, sublingual tolerance, intravenous tolerance, and low dose tolerance. In some embodiments of the invention allergens are added together with prebiotics/probiotics. Said allergens may be introduced in naïve form. In some embodiments antigens may be pulverized, chemically altered, denatured, partially denatured, or roasted. In some particular embodiments the allergy treated is acquired as a result of dysbiosis. Said dysbiosis may be the result of environmental changes, stress, or as result of antibiotic treatment.

For the purpose of illustrating the invention, various terms are provided below. These are merely as a guide and not to be restrictive.

“Allergen”: An “allergen” is an antigen that (i) elicits an IgE response in an individual; and/or (ii) elicits an asthmatic reaction (e.g., chronic airway inflammation characterized by eosinophilia, airway hyperresponsiveness, and excess mucus production), whether or not such a reaction includes a detectable IgE response. Preferred allergens for the purpose of the present invention are protein allergens, although the invention is not limited to such. An exemplary list of protein allergens is presented as an Appendix in U.S. patent application (U.S. Ser. No. 09/455,294), filed Dec. 6, 1999, which is incorporated herein by reference. This list was adapted on Jul. 22, 1999, from ftp://biobase.dk/pub/who-iuis/allergen.list, which provides lists of known allergens. Allergens from plants may be subdivided in allergens from pollen and the like and allergens from seeds. Allergens from seeds are preferably storage proteins such as 2S-albumin or conglutin. In purified form such storage proteins are, in a preferred embodiment, for instance, Ara h 2 or Ara h 6 from peanut. Alternatively, allergens from plants may be subdivided in allergens from fruit, such as lipid transfer proteins, allergens from oil crops, such as peanut or soybean, and allergens from tree nuts and seeds such as hazelnut, walnut and sunflower seed. Allergens from insects are preferably venoms from for instance bee or wasp, which may be purified to obtain individual allergens. Allergens that can be used with the teachings herein non-exclusively include food allergens such as those derived from: a) peanuts; b) wheat; c) soy; d) fish; e) shellfish; f) tree nuts; g) dairy, and h) eggs. Further environmental allergens that can be used with the teachings herein can be derived from a source selected from the group consisting of: a) animal dander, b) pollen, c) grass, d) mold, e) insect stings, f) dust mites, g) latex, and h) animal saliva. Animal dander and saliva can be allergens derived from sources such as pet and livestock animals including cats, dogs, horses, sheep and the like.

“Allergic reaction”: An allergic reaction is a clinical response by an individual to an antigen. Symptoms of allergic reactions can affect the cutaneous (e.g., urticaria, angioedema, pruritus), respiratory (e.g., wheezing, coughing, laryngeal edema, rhinorrhea, watery/itching eyes), gastrointestinal (e.g., vomiting, abdominal pain, diarrhea), and/or cardiovascular (if a systemic reaction occurs) systems. For the purposes of the present invention, an asthmatic reaction is considered to be a form of allergic reaction.

“Anaphylactic antigen”: An “anaphylactic antigen” according to the present invention is an antigen that is recognized to present a risk of anaphylactic reaction in allergic individuals when encountered in its natural state, under natural conditions. For example, for the purposes of the present invention, pollens and animal danders or excretions (e.g., saliva, feces, urine) are not considered to be anaphylactic antigens. On the other hand, some food antigens, insect antigens, drugs, and rubber (e.g., latex) antigens are generally considered to be anaphylactic antigens. Food antigens are particularly preferred anaphylactic antigens for use in the practice of the present invention. Particularly interesting anaphylactic antigens are those (e.g., peanuts, tree nuts, seeds, insect venom, seafood, shellfish, and fish) to which reactions are commonly so severe as to create a risk of death.

“Anaphylaxis” or “anaphylactic reaction”: “Anaphylaxis” or “anaphylactic reaction”, as used herein, refers to an immune response characterized by mast cell degranulation secondary to antigen-induced cross-linking of the high-affinity IgE receptor on mast cells with subsequent mediator release and the production of pathological responses in target organs, e.g., airway, skin, digestive tract, and cardiovascular system. As is known in the art, the severity of an anaphylactic reaction may be monitored, for example, by assaying cutaneous reactions, puffiness around the eyes and mouth, and/or diarrhea, followed by respiratory reactions such as wheezing and labored respiration. The most severe anaphylactic reactions can result in loss of consciousness and/or death.

“Animal”: The term animal, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). An animal may be a transgenic animal.

“Antigen”: An “antigen” is (i) any compound or composition that elicits an immune response; and/or (ii) any compound that binds to a T cell receptor (e.g., when presented by an MHC molecule) or to an antibody produced by a B-cell. Those of ordinary skill in the art will appreciate that an antigen may be a collection of different chemical compounds (e.g., a crude extract or preparation) or a single compound (e.g., a protein). Preferred antigens are protein antigens, but antigens need not be proteins for the practice of the present invention. Other preferred antigens are small molecules such as drugs (e.g., penicillin).

“Antigen presenting cell”: An APC is any cell that is capable of presenting antigen in a manner sufficient to induce an immune response in a naive cell or to stimulate an immune response in a previously primed cell. A “professional” APC (pAPC) is an APC that displays antigen in the context of an MHC molecule and (i) is capable of providing co-stimulatory signals and initiating a primary immune response (i.e., activating or priming a naive T cell); and/or (ii) expresses cytokines sufficient to induce an immune response in a committed T cell. Such pAPCs include macrophages, dendritic cells, and B cells.

“Peptide”: According to the present invention, a “peptide” comprises a string of at least three amino acids linked together by peptide bonds. Peptide may refer to an individual peptide or a collection of peptides. Inventive peptides preferably contain only natural amino acids, although non-natural amino acids (i.e., compounds that do not occur in nature but that can be incorporated into a polypeptide chain; see, for example,

http://www.cco.caltech.edu/.about.dadgrp/Unnatstruct.gi-f, which displays structures of non-natural amino acids that have been successfully incorporated into functional ion channels) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofamesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Th1 response” and “Th2 response”: Th1 and Th2 responses are well-established alternative immune system responses that are characterized by the production of different collections of cytokines and/or cofactors. For example, Th1 responses are generally associated with the production of cytokines such as IL-1, IL-2, IL-12, IL-18, IFN, IFN, TNF, etc.; Th2 responses are generally associated with the production of cytokines such as IL-4, IL-5, IL-10, etc. The extent of T cell subset suppression or stimulation may be determined by any available means including, for example, intra-cytoplasmic cytokine determination. In preferred embodiments of the invention, Th2 suppression is assayed, for example, by quantitation of IL-4, IL-5, and/or IL-13 in stimulated T cell culture supernatant or assessment of T cell intra-cytoplasmic (e.g., by protein staining or analysis of mRNA) IL-4, IL-5, and/or IL-13; Th1 stimulation is assayed, for example, by quantitation of IFN, IFN, IL-2, IL-12, and/or IL-18 in activated T cell culture supernatant or assessment of intra-cytoplasmic levels of these cytokines.

For use in the practice of the invention, allergens may be any antigen to which an individual may be sensitive to is relevant to this invention. Preferred antigens include protein antigens, and of particular interest are anaphylactic protein antigens. Anaphylactic antigens include food antigens, insect antigens, and rubber antigens (e.g., latex). In particular, peanut and tree nut (e.g., walnut, almond, pecan, cashew, hazelnut, pistachio, pine nut, brazil nut) antigens, dairy (e.g., egg, milk) antigens, seed (e.g., sesame, poppy, mustard) antigens, fish/shellfish (e.g., cod, shrimp, crab, lobster, clams) antigens, and insect antigens are anaphylactic antigens according to the present invention. Particularly preferred anaphylactic antigens are food antigens; peanut (e.g., Ara h 1-3), milk, egg, and fish/shellfish (e.g., tropomyosin) antigens are especially preferred. In some cases, it will be desirable to work in systems in which a single compound (e.g., a single protein) is responsible for most observed allergies. In other cases, the invention can be applied to more complex allergens. In a particularly preferred embodiment, the antigen has predominately linear epitopes.

In some embodiments, the antigen may be administered in association with cytokines, adjuvants, inducting agents, or other immunomodulatory substances. In some embodiments of the invention, a protein antigen is provided by a polynucleotide encoding the antigen. DNA or RNA may be used in the invention; however, DNA is generally preferred given its greater stability. The polynucleotide may be provided in the context of a delivery vector such as a plasmid or virus. Preferably, the polynucleotide includes expression sequences (e.g., promoter, enhancer, splicing signals, Shine-Delgarno sequence, etc.) sufficient to direct protein expression in the relevant individual. A wide variety of such sequences is known in the art (Sambrook et al. Molecular Cloning. A Laboratory Manual, 2nd Ed., 1989; Miller & Calos, eds., Gene Transfer vectors for Mammalian Cells, 1987; Ausubel et al., eds., Current Protocols in Molecular Biology, 1987; each of which is incorporated herein by reference). Once inside a cell, the polynucleotide is transcribed and translated in order to produce the protein antigen in situ. Production of the protein antigen leads to sensitization of the individual. In a preferred embodiment, the expression system (e.g., promoter, enhancer, splicing signals, etc.) and vector are matched to the species to which it is being administered. For example, if a mammal such as a mouse was to be treated using immunotherapy, the promoter used to drive protein production might be the cytomegalovirus (CMV) promoter. Modified versions of the antigens may also be used in the present invention. Any type of modification can be used. They may be biological or chemical. The antigen may contain unnatural amino acids; may be modified, e.g., glycosylated, phosphorylated, hydroxylated, etc., may lack modification; may be cross-linked; may contain mutations (e.g., substitutions, deletions); etc. Antigens may also be a fusion protein (e.g., fused with a cytokine, another antigen, the same antigen, an inducing factor, an adjuvant, etc.)

In one embodiment of the invention, allergies treated are allergies that the patient possessed since birth or since a period of longer than one year prior to the date of intervention. In another embodiment of the invention, allergies treated are allergies that have been induced by exposure to one or multiple agents. In some embodiments, the invention teaches the use of prebiotics and/or probiotics in order to treat allergies that have been induced by exposure to an antibiotic or one or more agents that have induced damage on the gut microflora. In one embodiment of the invention, restoration of the gut microflora is performed in order to increase propensity for tolerance induction. In one embodiment tolerance induction refers to oral tolerance. In other embodiments, tolerance induction refers to sublingual tolerance. In another embodiment, tolerance induction refers to tolerance induced by epicutaneous immunotherapy. In another embodiment, tolerance induction refers to tolerance induced by subcutaneous immunotherapy with modified allergen. In another embodiment, tolerance refers to a state of immunological unresponsiveness induced by administration of lysosomal-associated membrane protein (LAMP)-DNA based vaccines.

In the practice of the invention, oral tolerance is induced by ingestion of food allergens at doses lower than those capable of triggering allergic reactions and disease are escalated under supervision of a physician. Examples from the literature are provided to guide one of skill in the art to practice oral tolerance in the presence of prebiotic/probiotic manipulation of the gut microbiome. In one publication 4 peanut-allergic children underwent oral immunotherapy as daily doses of peanut flour increasing from 5 to 800 mg of protein with 2-weekly dose increases. After 6 further weeks of treatment, the oral challenge was repeated to define change in dose threshold and subjects continued daily treatment. Preintervention challenges confirmed peanut allergy and revealed dose thresholds of 5-50 mg (1/40-1/4 of a whole peanut); one subject had anaphylaxis during challenge and required adrenaline injection. All subjects tolerated immunotherapy updosing to 800 mg protein and i.m. adrenaline was not required. Each subject tolerated at least 10 whole peanuts (approximately 2.38 g protein) in postintervention challenges, an increase in dose threshold of at least 48-, 49-, 55- and 478-fold for the four subjects [9]. In another oral tolerance study, 23 children with IgE-mediated peanut allergy that was confirmed by positive double-blind, placebo-controlled food challenge (DBPCFC) received oral immunotherapy following a rush protocol with roasted peanut for 7 days. If a protective dose of at least 0.5 g peanut was not achieved, patients continued with a long-term buildup protocol using biweekly dose increases up to at least 0.5 g peanut. A maintenance phase for 8 weeks was followed by 2 weeks of peanut avoidance and a final DBPCFC. It was found that patients tolerated a median dose of only 0.15 g peanut. Twenty-two of 23 patients continued with the long-term protocol. After a median of 7 months, 14 patients reached the protective dose. At the final DBPCFC, patients tolerated a median of 1 g (range, 0.25-4 g) in comparison with 0.19 g peanut at the DBPCFC before OIT (range, 0.02-1 g). In 2.6% of 6137 total daily doses, mild to moderate side effects were observed; in 1.3%, symptoms of pulmonary obstruction were detected. OIT was discontinued in 4 of 22 patients because of adverse events. There was a significant increase in peanut-specific serum IgG(4) and a decrease in peanut-specific IL-5, IL-4, and IL-2 production by PBMCs after oral immunotherapy [10]. In another example, a 4 year old child with severe peanut allergy (age 4 years, facial urticaria and lip angioedema upon licking a peanut; peanut skin prick test, 13×10 mm; specific immunoglobulin (Ig) E>100 kUA/L) was treated determined to have a threshold oral challenge dose that was 62.5 mg. Several peanut solutions were prepared and sequentially administered at the patient's home. Over 138 days, the dose was increased from 0.625 to 5500 mg. There were 43 mild-to-moderate reactions (28% of the doses administered). Oral tolerance was induced successfully [11]. The previous mentioned clinical trials are provided as a guide to dosages and protocols that can be applied to the current invention. The innovative step in the current invention teaches that modification of the microbiota may be useful for enhancing tolerogenic processes, as well as desensitizing patients to allergens. In one particular embodiment amelioration of dysbiosis is used to treat an allergy that has occurred subsequent to an event that induced dysbiosis. In one embodiment said event inducing dysbiosis is treatment with an antibiotic. In another event, said dysbiosis is caused by a stressful event. In embodiment the utilization of probiotics is provided alone, in another embodiment the probiotics are administered together with allergens of interest in order to induce an antigen specific tolerogenic effect. In another embodiment said probiotics are administered with prebiotics to enhance the ability of said probiotics to exert an alternation in the microbiota. The invention envisions the utilization of microbiota modulation as a means of enhancing known methods of allergen desensitization, some of these methods are listed below. In OIT, native or modified food allergen is ingested; whereas in SLIT, liquid allergen extract is applied under the tongue. SLIT and OIT respectively start with sub-threshold doses in 0.0001 μg- and 0.1 mg-range, which are increased under physician supervision during an initial rapid dose-escalation day up to 0.01 μg-range and mg-range. The highest tolerated dose after initial dose-escalation may be repeated on the following day to confirm it will be safely tolerated during daily doses at home. This is followed by a build-up phase during which daily doses are increased every 2 weeks under physician supervision up to maintenance doses in mg-range for SLIT and gram-range for OIT. Daily maintenance dosing is continued for months to years.

In one embodiment, probiotic bacteria are administered at a concentration of 10⁽⁶⁾ to 10⁽¹²⁾ colony forming units (CFU) of bacteria per gram of support material, and more particularly from 10⁽⁸⁾ to 10⁽¹²⁾ CFU of bacteria/gram of support material, preferably 10⁽⁹⁾ to 10⁽¹²⁾ CFU/gram of support material for the lyophilized form. In the specific embodiment, said bacterium is administered orally, at a frequency sufficient to induce immune modulation. Specifically, immune modulation comprises upregulation of Th1 cytokines, and/or reduction in Th2 cytokines, and/or augmentation of regulatory T and/or regulatory B cells. In one embodiment, allergen of interest is administered subsequent to initiation of probiotic treatment. In another embodiment, allergen of interest is administered prior to initiation of probiotic treatment, in a preferred embodiment, administration of allergen of interest is administered concurrently with probiotic treatment.

Suitably the probiotic bacteria used in accordance with the present invention may be administered at a dosage of from about 10⁽⁶⁾ to 10⁽¹²⁾ CFU of microorganism/dose, preferably about 10⁽⁸⁾ to 10⁽¹²⁾ CFU of microorganism/dose. By the term “per dose” it is meant that this amount of microorganism is provided to a subject either per day or per intake, preferably per day. For example, if the microorganism is to be administered in a food product (for example in a yoghurt)—then the yoghurt will preferably contain from about 10⁽⁸⁾ to 10⁽¹²⁾ CFU of the microorganism. Alternatively, however, this amount of microorganism may be split into multiple administrations each consisting of a smaller amount of microbial loading—so long as the overall amount of microorganism received by the subject in any specific time (for instance each 24 hour period) is from about 10⁽⁶⁾ to about 10⁽¹²⁾ CFU of microorganism, preferably 10⁽⁸⁾ to 10⁽¹²⁾ CFU of microorganism.

In accordance with the present invention an effective amount of at least one strain of a microorganism may be at least 10⁽⁶⁾ CFU of microorganism/dose, preferably from about 10⁽⁶⁾ to about 10⁽¹²⁾ CFU of microorganism/dose, preferably about 10⁽⁸⁾ to about 10⁽¹²⁾ CFU of microorganism/dose. In one embodiment, preferably the bacteria may be administered at a dosage of from about 10⁽⁶⁾ to about 10⁽¹²⁾ CFU of microorganism/day, preferably about 10⁽⁸⁾ to about 10.sup.12 CFU of microorganism/day. Hence, the effective amount in this embodiment may be from about 10⁽⁶⁾ to about 10⁽¹²⁾ CFU of microorganism/day, preferably about 10⁽⁸⁾ to about 10⁽¹²⁾ CFU of microorganism/day.

The probiotic mixtures may be used according to the present invention in the form of solid or liquid preparations or alternatives thereof. Examples of solid preparations include, but are not limited to tablets, capsules, dusts, granules and powders which may be wettable, spray-dried or freeze-dried. Examples of liquid preparations include, but are not limited to, aqueous, organic or aqueous-organic solutions, suspensions and emulsions. Suitable examples of forms include one or more of: tablets, pills, capsules, ovules, solutions or suspensions, which may contain flavouring or colouring agents, for immediate-, delayed-, modified-, sustained-, pulsed- or controlled-release applications. By way of example, if the composition of the present invention is used in a tablet form—such for use as a functional ingredient—the tablets may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycoliate, croscarmellose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc may be included. Furthermore, examples of nutritionally acceptable carriers for use in preparing the forms include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethylcellulose, polyvinylpyrrolidone, and the like. Preferred excipients for the forms include lactose, starch, a cellulose, milk sugar or high molecular weight polyethylene glycols.

In one embodiment, probiotic bacteria are administered in the form of a nutraceutical. Nutraceuticals, whether in the form of a liquid extract or dry composition, are edible and may be eaten directly by humans or mammals. Said nutraceuticals are preferably provided to humans in the form of additives or nutritional supplements e.g., in the form of tablets of the kind sold in health food stores, or as ingredients in edible solids, more preferably processed food products such as cereals, breads, tofu, cookies, ice cream, cakes, potato chips, pretzels, cheese, etc., and in drinkable liquids e.g., beverages such as milk, soda, sports drinks, and fruit juices. Thus, in one embodiment a method is provided for enhancing the nutritional value of a food or beverage by intermixing the food or beverage with a nutraceutical in an amount that is effective to enhance the nutritional and probiotic or immune modulatory value of the food or beverage. In one embodiment, a flavoring agent is added. Preferred flavoring agents include sweeteners such as sugar, corn syrup, fructose, dextrose, maltodextrose, cyclamates, saccharin, phenyl-alanine, xylitol, sorbitol, maltitol, and herbal sweeteners such as Stevia. Examples of foods into which probiotics useful for the practice of the invention can be incorporated into include soft drinks, a fruit juice or a beverage comprising whey protein, health teas, cocoa drinks, milk drinks and lactic acid bacteria drinks.

Probiotic bacteria may be administered together with agents known to enhance efficacy and retention of probiotics, including various prebiotics such as fructo-oligosaccharides [12-14], galacto-oligosaccharides [15, 16], arabinogalactan [17, 18], and inulin [19].

In a further embodiment of the present invention various extracts and plant powders are incorporated into our compositions, depending on the desired properties according to the end use of said compositions. These compositions according to the present invention can be characterized in that in addition to the discussed prebiotics and phytosterols and lecithins the said further plant extracts or powders are one or more of those of Panax ginseng (red, Korean ginseng), Panax ginseng (white, Chinese ginseng), Rhodiola rosea (golden root), Panax quinquefolium (American ginseng), Eleutherococcus senticosus (Siberian ginseng), Cynara scolymus (artichoke), Uncaria tomentosa (Cat's claw), Lepidium meyenii (maca, Peruvian ginseng), Paullinia cupana (guarana), Croton lechleri (Sangre de Grado), Whitania somnifera (ashwagandha, Indian ginseng), Panax japonicus (Japanese ginseng), Panax vietnamensis (Vietnamese ginseng), Panax trifolius, Panax pseudoginseng, Panax notoginseng, Malpighia glabra (acerola), Ylex paraguayiensis (Yerba mate), Astragalus membranaceus (astragalus), Stevia rebaudiana (stevia), Pfaffia paniculata (Brazilian ginseng, suma), Ginkgo biloba, Tabebuia impetiginosa (Pau d'arco), Echinacea purpurea, Peumus boldus (boldo), Gynostemma pentaphyllum (Jiaogulan, also known as Southern Ginseng or Xiancao), Sutherlandia frutescens (African ginseng), Aloe vera (aloe), Cistanche salsa, Cistanche deserticola (and other Cistanche sp.), Codonopsis pilosula (“poor man's ginseng.”), Nopal opuntia (Prickly pear cactus), Citrus sinensis (Citrus aurantium) and other members of the citrus family (lemon, lime, tangerine, grapefruit), Camelia sinensis (tea), Plantago psyllium (psyllium), Amaranth edulis and other amaranth sp. (amaranth), Commiphora mukul (guggul lipid), Serenoa repens, Serenoa serrulata (saw palmetto), Cordyceps sinensis (Cordycaps), Lentinula edodes (Shitake), Ganoderma lucidium (Reishi), Grifola frondosa (maitake), Tremella fuciformis (Silver ear), Poria cocos (Hoelen), Hericium erinaceus (Lion's Mane), Agaricus blazei (Sun mushroom), Phellinus linteus (Mulberry yellow polypore), Trametes versicolor, Coriolus versicolor (Turkey tails), Schizophyllum commune (Split gill), Inonotus obliquus (Cinder conl), oat bran, rice bran, linseed, garlic, Ceratonia siliqua (locust been gum or flour from the seeds of carob tree), Cyanopsis tetragonoloba (guar gum, EU Food additive code E412), Xanthomonas campestris (xanthan gum). These plant extracts and plant powders are capable to potentiate the bioactivity of these compositions based on prebiotics, phytosterols, lecithins, vitamins and minerals. In given cases it also adds other prebiotics to the aforementioned prebiotic mixtures. These can result in more pronounced bioactivities as prebiotics and also in the chosen other bioactivity directions.

The nutraceuticals described herein are intended for human consumption and thus the processes for obtaining them are preferably conducted in accordance with Good Manufacturing Practices (GMP) and any applicable government regulations governing such processes. Especially preferred processes utilize only naturally derived solvents. In contrast to nutraceuticals, the so-called “medical foods” are not meant to be used by the general public and are not available in stores or supermarkets. Medical foods are not those foods included within a healthy diet to decrease the risk of disease, such as reduced-fat foods or low-sodium foods, nor are they weight loss products. A physician prescribes a medical food when a patient has special nutrient needs in order to manage a disease or health condition, and the patient is under the physician's ongoing care. The label must clearly state that the product is intended to be used to manage a specific medical disorder or condition. An example of a medical food is nutritionally diverse medical food designed to provide targeted nutritional support for patients with chronic inflammatory conditions. Active compounds of this product are for instance one or more of the compounds described herein. The present invention thus relates to the use of an immuno-modulating properties of probiotics as related to prevention and/treatment of pregnancy complications. Thus said probiotics can be used in the preparation of a medicament, a vaginal suppository, medical food or nutraceutical to induce immune tolerance or immune modulation.

In some embodiments, the compositions according to the present invention comprise prebiotic components selected from fructose polymers GF.sub.n and F.sub.m, either containing a glucose (G) end-group, or without this glucose end-group and one or more component of a group of prebiotics consisting of modified or unmodified starch and partial hydrolysates thereof, partially hydrolysed inulin, natural oligofructoses, fructo-oligosaccharides (FOS), lactulose, galactomannan and suitable partial hydrolysates thereof, indigestible polydextrose, acemannan, various gums, indigestible dextrin and partial hydrolysates thereof, trans-galacto-oligosaccharides (GOS), xylo-oligosaccharides (XOS), beta-glucan and partial hydrolysates thereof, together if desired with phytosterol/phytostanol components and their suitable esters, and if desired other plant extracts, mineral components, vitamins and additives. The fructose polymers of GF.sub.n or F.sub.m structures (G=glucose; F=fructose; n>2; m>2) are linear fructose polymers having either a glucose (G) and -group, or being without this glucose and -group. Oligofructoses are consisted of 3 to 10 carbohydrate units. Above that, chicory inulin contains 10 to 60 carbohydrate units, typically with 27 carbohydrates (fructoses with our without one glucose end-group and a fructose chain). Other plants may produce different fructans. These fructans are capable to increase the number of colonized and planktonic bacteria in the large intestine. This results in a change that those bacteria that are less advantageous or may turn dangerous are suppressed by the higher probiotic colony of bacteria. Depending on the chain length of these fructans or other prebiotics, they can be fermented by probiotic bacteria at different positions in the colon. We have found that the longer inulins are capable to rich the distal colon and sigmoid colon and exert their anticancer actions in the positions where typically most of the cancerous problems occur. The occurrence of these cancers can be the result of various types of carcinogenesis. It has been demonstrated in the literature that directly induced chemical carcinogenesis can be greatly reduced by probiotic bacteria. The prebiotic compositions of our invention can corroborate this effect by considerably increasing the number of Bifidocateria and other beneficial probiotic strains. The local chemical carcinogenesis can also be the result of the formation of secondary bile acids. These secondary bile acids are often formed upon the action of enzymes produced by resident Clostridia. By probiotic suppression of the number of these bacteria according to the invention, the chance of secondary bile acid formation can also be reduced. This can be demonstrated by measuring the faecal primary/secondary bile acid ratio. Other prebiotics can be selected from a group of prebiotics consisting of various gums (guar gum, xanthan gum, locust been gum), carob seed flour, oat bran, rice bran, barley, modified or unmodified starch and suitable partial hydrolysates thereof, partially hydrolysed inulin, natural or synthetic/biosynthetic oligofructoses, fructo-oligosaccharides (FOS), lactulose, galactomannan and suitable hydrolysates thereof, indigestible polydextrose, indigestible dextrin and partial hydrolysates thereof, trans-galacto-oligosaccharides (GOS), xylo-oligosaccharides (XOS), acemannan, lentinan or beta-glucan and partial hydrolysates thereof, polysaccharides P and K (PSP, PSK), tagatose, various fungal oligosaccharides and polysaccharides, together with other components.

In some embodiments of the invention, probiotic strains are screened for ability to induce oral tolerance and/or to enhance the process of oral tolerance towards allergens. In one embodiment probiotic bacteria screening is accomplished by assessing ability of said probiotics or products thereof, to induce production of cytokines inhibitory to allergy in vitro. Such in vitro systems include culturing of said probiotics, or products thereof, in the presence of immune cells and subsequently assessing ability to induce production of cytokines including IL-2, IL-10, TGF-beta, and interferon gamma. Bacteria of various species may be assessed, including lactobacillus species, e.g., Lactobacillus acidophilus, L. plantarum, L. casei, L. rhamnosus, L. delbrueckii (including subspecies bulgaricus), L. reuteri, L. fermentum, L. brevis, L. lactis, L. cellobiosus, L. GG, L. gasseri, L. johnsonii, and L. plantarum; bifidobacterium species, e.g., Bifidobacterium bifidum, B. infantis, B. longum, B. thermophilum, B. adolescentis, B. breve, B. animalis; streptococcus species, e.g., Streptococcus lactis, S. cremoris, S. salivarius (including subspecies thermophilus), and S. intermedius; Leuconostoc species; Pediococcus species; Propionibacterium species; Bacillus species; non-enteropathogenic Escherichia species, e.g., non-enteropathogenic Escherichia coli, e.g., E. coli Nissle, and the like; and Enterococcus species such as Enterococcus faecalis, and E. faecium. The compounds can also be in micro-encapsulated form with one or more excipients as noted above. The solid dosage forms of tablets, dragees, capsules, pills and granules can be prepared with coatings and shells such as enteric coatings, release controlling coatings, and other coatings well known in the pharmaceutical formulating art. In such solid dosage forms the active compound may be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

In order to determine the amount of allergens needed to reduce or prevent allergic reactions to allergenic proteins, or to exhibit a detectable therapeutic or preventative effect, various protocols may be used. The effect can be detected by, for example, reduced IgE levels to the allergenic protein and increased thresholds for challenge by allergens. The precise effective amount for a subject will depend upon the subject's size and health, the nature and extent of the condition, and the therapeutics or combination of therapeutics selected for administration. In case a subject has undergone treatment with antihistamines, dosages will typically tend to be higher than without such pre-treatment. Thus, it is not useful to specify an exact effective amount in advance. However, the effective amount for a given situation can be determined by routine experimentation and is within the routine judgment of the clinician or experimenter. Specifically, the compositions of the present invention can be used to reduce or prevent allergic reactions to allergenic proteins and/or accompanying biological or physical manifestations. Such manifestations may include contraction of smooth muscle in the airways or the intestines, the dilation of small blood vessels and the increase in their permeability to water and plasma proteins, the secretion of thick sticky mucus, and, in the skin, redness, swelling and the stimulation of nerve endings that results in itching or pain. Manifestations that may be prevented by immunotherapy according to the present invention include skin manifestations such as rashes, hives or eczema; gastrointestinal manifestations including cramping, nausea, vomiting or diarrhoea; or respiratory manifestations including sneezing or runny nose, coughing, wheezing or shortness of breath. Other manifestations that may be prevented include itching of skin, flushes, congestion, eye irritation, asthma, itching in the mouth or throat which may progress to swelling and anaphylaxis. Methods that permit the clinician to establish initial immunotherapy dosages are known in the art (e.g. U.S. Pat. No. 4,243,651). The dosages to be administered must be safe and efficacious. As with any medical treatment, a balance must be struck between efficacy and toxicity.

For purposes of the present invention, an effective dose will be from about 0.1 ng/kg to 0.1 mg/kg, 10 ng/kg to about 10 μg/kg, or 0.1 μg/kg to 1 μg/kg of the modified allergenic protein relative to the body weight of the individual to which it is administered. Often, a treatment will comprise starting with the administration of dosages at the lower end of these ranges and increasing the dosages as the treatment progresses. These dosages are intended for modified allergens obtained from purified allergens. For modified allergens based on a crude extract of the allergen, dosages may be higher corresponding to the purity of the extract used. For typical desensitization treatment, it is typically necessary for the patient to receive frequent administrations, e.g., initially every two or three days, gradually reducing to once every two or three weeks. Other suitable desensitisation programs include subcutaneous injections once every 2-4 weeks the dosage of which injections may gradually increase over a period of 3-6 months, and then continuing every 2-4 weeks for a period of up to about 5 years. It is also possible, particular for sublingual administration, that daily administrations are given.

Desensitization protocols may also comprise a form of treatment conventionally known in various equivalent alternative forms as rapid desensitization, rapid allergen immunotherapy, rapid allergen vaccination, and rapid or rush immunotherapy. In broad terms, this procedure aims to advance an allergic patient to an immunizing or maintenance dose of extract (i.e., allergen) by administering a series of injections (or via another suitable carrier) of increasing doses of the allergen at frequent (e.g. hourly) intervals. If successful, the patient will exhibit an improved resistance to the allergen, possibly even presenting a total non-reactivity to any subsequent allergen exposure. Various desensitization protocols are known in the art and may for instance comprise a method of treating a patient having an immediate hypersensitivity to an allergen using an accelerated rapid immunotherapy schedule in combination with a method of pre-treating such patient with prednisone and histamine antagonists prior to receiving the accelerated immunotherapy such as described in US patent application 2003/082212.

As discussed above, the present invention provides modified allergens which have biological properties which make them of interest for the treatment of allergies and in particular anaphylactic reactions. Accordingly, in another aspect of the present invention, pharmaceutical compositions are provided, wherein these compositions comprise a modified allergen, and optionally comprise a pharmaceutically acceptable carrier and/or an adjuvant. It will be appreciated that certain of the modified allergens of present invention can exist in free form for treatment or may be provided as crude preparations, such as a chemical or proteolytic digestion of a food extract (see, for example, Hong et al., J. Allergy Clin. Imunol. 104:473, 1999). Those of ordinary skill in the art will also appreciate that inventive modified allergens may be provided by combination or association with one or more other agents such as targeting agents or may be encapsulated (e.g., within a liposome, nanoparticle, or a live, preferably attenuated, infectious organism such as a bacterium or a virus).

In some embodiments of the invention, it is desirable to inactivate probiotic bacteria prior to administration. Inactivation may be achieved by means including irradiation. The probiotic bacteria are irradiated at an energy and for a period of time sufficient to inhibit bacterial growth in vitro and/or to render the probiotic bacteria non-viable, e.g., such that growth in in vitro culture is undetectable using standard methods. In some embodiments, the irradiation is ionizing radiation. Gamma radiation is an example of ionizing radiation. For example, the bacteria are irradiated using gamma irradiation in an amount of from about 5 kiloGray (kGy) to about 50 kGy, from about 10 kGy to about 20 kGy, from about 20 kGy to about 40 kGy, or from about 25 kGy to about 35 kGy. Bacteria are irradiated for a period of time of from about 15 seconds to about 48 hours, e.g., from about 15 seconds to about 1 minute, from about 1 minute to about 15 minutes, from about 15 minutes to about 30 minutes, from about 60 minutes to about 90 minutes, from about 90 minutes to about 2 hours, from about 2 hours to about 4 hours, from about 4 hours to about 8 hours, from about 8 hours to about 12 hours, from about 12 hours to about 16 hours, from about 16 hours to about 24 hours, from about 24 hours to about 36 hours, or from about 36 hours to about 48 hours. The total amount of irradiation and the duration of irradiation can be adjusted, depending on various factors, e.g., the number of bacteria being irradiated. The total amount of irradiation and the duration of irradiation that results in bacteria that have reduced viability or are non-viable (e.g., are unable to grow in in vitro culture) are readily determined by those of ordinary skill in the art. In other embodiments, the radiation is ultraviolet (UV) radiation. For example, the probiotic bacteria are exposed to UV radiation of from about 2000 μW sec/cm² to about 1,000 μW sec/cm². In some embodiments, the probiotic bacteria are inactivated by pasteurization. The process of pasteurization is well known in the art of food sciences. Any method for pasteurization can be used for the current invention. Pasteurization generally involves heating the material to be pasteurized at one of the following temperatures, for the following time period: at about 60° C. for at least about 30 minutes; at 72° C. for at least about 15 seconds; at 88° C. for at least about 1 second; at 90° C. for at least about 0.5 second; at 94° C. for about 0.1 second; at 96° C. for about 0.05 second; or 100° C. for about 0.01 second. Standard pasteurization conditions are found in the literature, e.g., in U.S. Pat. Nos. 6,475,545, 4,438,147, and 6,528,085. For example, in the present invention, pasteurization of liquids or solids comprising a suitable probiotic bacterium is carried out by heating the liquid or solid under conventional pasteurization conditions such as, for example, but not limited to, about 72° C. to about 85° C. for from about 15 seconds to about 10 minutes, e.g., about 72° C. to about 85° C. for from about 15 seconds to about 30 seconds, from about 20 seconds to about 40 seconds, from about 30 seconds to about 60 seconds, from about 1 minute to about 2 minutes, from about 2 minutes to about 5 minutes, or from about 5 minutes to about 10 minutes. Generally, temperatures above 90° C. are not used to inactivate bacteria in the present invention.

In some embodiments of the invention, administration of an inventive microbiome reparative oral tolerization composition can protect an individual against, subsequent inadvertent or intentional exposures to antigen. Thus, prior to exposure, or risk of exposure, to the allergic antigen may receive (e.g., by self-administration or through administration by a friend, relative, a acquaintance, or a healthcare professional, an inventive blocking agent. In another aspect of the present invention, administration of the inventive agent is followed by subsequent exposure to the antigen, or portions thereof, in the hope of eliciting tolerance to the antigen. In certain preferred embodiments, antigen exposure takes the form of standard immunotherapy or rush immunotherapy. Rush immunotherapy is typically performed to achieve tolerance to an antigen and is done with more antigen than is typically used in standard immunotherapy. Preferably, exposure to the antigen occurs within the passive desensitization period. For example, in many preferred embodiments, antigen exposure occurs within 48 hours, more preferably within 24 hours, and most preferably within 4-8 hours or less. Exposure to antigen may also involve exposure to cytokines, adjuvants, or inducing agents. Where exposure is to standard or rush immunotherapy, it preferably occurs in a hospital setting or other suitably equipped medical facility.

In the practice of the invention the treatment of an allergic disorder involves administering a subject formulation to an individual who is sensitized to an antigen (e.g., an allergen). The formulation of the invention is administered in an amount effective to treat the allergic disorder, e.g., to reduce production of IgE specific for the antigen (e.g., the allergen); to reduce the severity of a symptom of the allergic disorder; to reduce the amount of a conventional therapeutic agent that is required to treat the disorder; to reduce the frequency and/or severity of an allergic reaction to the allergen; and the like. Thus, an effective amount of a subject formulation is an amount that reduces the severity of a symptom and/or reduces a measurable parameter associated with the allergic disorder by at least about 10%, at least about 20%, at least about 25%, at least about 30%, at least about 40%, at least about 50%, at least about 60%, at least about 70%, at least about 80%, or at least about 90% or more, when compared with the symptom (e.g., the severity of the symptom), or when compared with the measurable parameter associated with the allergic disorder, in the absence of treatment with a subject formulation. In some embodiments, an effective amount of a subject formulation reduces the level of serum IgE in an individual by at least about 10%, at least about 20%, at least about 25%, at least about 30%, at least about 40%, at least about 50%, at least about 60%, at least about 70%, at least about 80%, or at least about 90% or more, when compared with the level of serum IgE in the absence of treatment with a subject formulation. In some embodiments, an effective amount of a subject formulation reduces the severity of symptoms (e.g., reduces the frequency of coughing, sneezing, wheezing, etc.) by at least about 10%, at least about 20%, at least about 25%, at least about 30%, at least about 40%, at least about 50%, at least about 60%, at least about 70%, at least about 80%, or at least about 90% or more, when compared with the frequency of coughing, sneezing, wheezing, etc. in the absence of treatment with a subject formulation.

### EXAMPLES

### Example 1

Patients are diagnosed with allergy using the “double blind placebo controlled food challenge”. This diagnostic test is considered the best way (gold standard) to prove or rule out allergy to a specific food. Double blind means that neither the person eating the preparation nor the health professional giving the food preparation know if the “suspected” food is an ingredient or not. The food preparations are very well “disguised” to mask texture, smell, taste or any other hints that may tell the ingredients. Placebo controlled means that this test is performed in two days at least: one of the days the patient receives the suspected food in the preparation, and the other day, a similar preparation is given but without the suspected allergen. This is done in a random sequence. Reactions are compared, and depending on symptoms and signs, the diagnosis of proven food allergy or not is made [20, 21]. Subsequent to diagnosis patients are treated with a regimen designed to ameliorate gut microbiome dysbiosis. Various tolerance induction regiments may be used. In one embodiment oral tolerogenesis is induced using protocols known in the art or modifications to said protocols In one embodiment, subjects are instructed to continue a strict peanut-free diet and to keep a diary of any missed doses or adverse symptoms. An epinephrine auto-injector is provided to all subjects. The initial day escalation phase is performed at a qualified physician with appropriate emergency medications available. Dosing begins at 0.1 mg peanut protein or placebo; doses are approximately doubled every 30 minutes until 6 mg is reached or the subject has symptoms. The highest tolerated dose is the starting dose for the buildup phase and is given on the research unit the following day. Subjects not tolerating at least 1.5 mg are withdrawn from the treatment protocol. Subjects are instructed to ingest each dose mixed in a vehicle food daily. Subjects are advised to hold dosing if febrile or ill and to take all doses on a full stomach. Dosing is resumed at home if the subject missed less than three daily doses; subjects returned for an observed dose if 3-5 doses are missed. Subjects return every two weeks for approximately 44 weeks for dose escalations. Doses are increased by 50-100% until the 75 mg dose and were then increased by 25-33% until the 4000 mg maintenance dose is reached. After reaching the maintenance dose of peanut flour or placebo, subjects ingested the dose daily for one month and then returned for the first oral food challenge at week 48.

### Example 2

A 12 year old American boy went through dysbiosis during a visit to India where he was treated with antibiotics following an episode of severe fever and cold like symptoms. After coming to US, he experienced several allergies towards common allergens such as wheat, peanuts, broccoli, sesame seeds etc. The common symptoms were significant GI discomfort and pain episodes that could last for hours. After trying many different treatments of pain killers, fibers and other medicines all of which provided some relief but no complete remedy, he was treated with over the counter probiotics which do not require a medical prescription. Some of the probiotic strains that was given with regular food as allergens (wheat, milk, vegetables, nuts) were B. Infantis, L. Rhamnosus, L. Acidophillus and B. Longum. B. Infantis was given at 1×10⁹ colony counts daily for 14 days. L. Rhamnosus, L. Acidophilulus and B. Longum were given at 1×10⁹ CFU per day for 14 days. In addition to the probiotics, prebiotics in the form of natural vegetable fibers as well as over the counter available fibers containing inulin and oligosaccharides were also given to boost survival and expansion of the probiotics. After a two-week treatment, the signs of pain and discomfort were significantly diminished with increased ability to eat regular food. After six weeks of treatment, all the allergy signs were completely gone and boy was able to eat everything and his microbiome seemed to have been fully restored.

### Example 3

A 15 year old girl, who after taking antibiotics, developed significant intolerance/allergy towards wheat and gluten was given a week long treatment of a probiotic mixture with small doses of regular wheat based bread or bread products such as pizza. The probiotic mixture contained B. Longum, L. Rhamnosus and B. Infantis at 1×10⁹ CFU each. After just a week, she felt significant improvement in her ability to have wheat and gluten products without any major issues.

### Example 4

A 45 year old man had developed allergies to his pet after taking antibiotics. After taking a probiotic mixture containing several strains including B. Longum and L. Rhamnosus, at 50 billion CFU total, (one per day for 30 days) allergies were cleared. More specifically, the formulation contained L. Acidophilus, B. Lactis, L. plantarum, L. paracasei, L. casei, L. brevis, L. rhamnosus, B. breve, L. Salivarius, B. bifidum, B. longum, and L. gasseri. Since the man continued to live with his pet during probiotic treatment, he was continually exposed to the allergens and probiotic help to create a tolerance to this allergen.

### REFERENCES

1. Boehncke, W. H., et al., Identification of HLA-DR and -DQ alleles conferring susceptibility to pollen allergy and pollen associated food allergy. Clin Exp Allergy, 1998. 28(4): p. 434-41.
2. Caubet, J. C., et al., Potential non-T cells source of interleukin-4 in food allergy. Pediatr Allergy Immunol, 2014. 25(3): p. 243-9.
3. Remes, S. and P. Kulmala, The interplay between risk and preventive factors explains why some children develop allergies to certain foods and others show tolerance. Acta Paediatr, 2018.
4. Nwaru, B. I., et al., Prevalence of common food allergies in Europe: a systematic review and meta-analysis. Allergy, 2014. 69(8): p. 992-1007.
5. Branum, A. M. and S. L. Lukacs, Food allergy among children in the United States. Pediatrics, 2009. 124(6): p. 1549-55.
6. Sicherer, S. H., A. Munoz-Furlong, and H. A. Sampson, Prevalence of peanut and tree nut allergy in the United States determined by means of a random digit dial telephone survey: a 5-year follow-up study. J Allergy Clin Immunol, 2003. 112(6): p. 1203-7.
7. Sicherer, S. H., et al., US prevalence of self-reported peanut, tree nut, and sesame allergy: 11-year follow-up. J Allergy Clin Immunol, 2010. 125(6): p. 1322-6.
8. Sampson, H. A., et al., Food allergy: a practice parameter update-2014. J Allergy Clin Immunol, 2014. 134(5): p. 1016-25 e43.
9. Clark, A. T., et al., Successful oral tolerance induction in severe peanut allergy. Allergy, 2009. 64(8): p. 1218-20.
10. Blumchen, K., et al., Oral peanut immunotherapy in children with peanut anaphylaxis. J Allergy Clin Immunol, 2010. 126(1): p. 83-91 e1.
11. Ojeda, P., et al., Induction of oral tolerance to peanut: a successful home-based protocol. J Investig Allergol Clin Immunol, 2010. 20(6): p. 524-8.
12. Valdes-Varela, L., P. Ruas-Madiedo, and M. Gueimonde, In vitro fermentation of different fructo-oligosaccharides by Bifidobacterium strains for the selection of synbiotic combinations. Int J Food Microbiol, 2017. 242: p. 19-23.
13. Sanches Lopes, S. M., et al., Chemical characterization and prebiotic activity of fructo-oligosaccharides from Stevia rebaudiana (Bertoni) roots and in vitro adventitious root cultures. Carbohydr Polym, 2016. 152: p. 718-725.
14. Bryk, G., et al., Effect of a mixture of GOS/FOS((R)) on calcium absorption and retention during recovery from protein malnutrition: experimental model in growing rats. Eur J Nutr, 2016. 55(8): p. 2445-2458.
15. da Costa Ribeiro, H. J., et al., Normal Growth of Healthy Infants Born from HIV+ Mothers Fed a Reduced Protein Infant Formula Containing the Prebiotics Galacto-Oligosaccharides and Fructo-Oligosaccharides: A Randomized Controlled Trial. Clin Med Insights Pediatr, 2015. 9: p. 37-47.
16. Bruno-Barcena, J. M. and M. A. Azcarate-Peril, Galacto-oligosaccharides and Colorectal Cancer: Feeding our Intestinal Probiome. J Funct Foods, 2015. 12: p. 92-108.
17. Terpend, K., et al., Arabinogalactan and fructo-oligosaccharides have a different fermentation profile in the Simulator of the Human Intestinal Microbial Ecosystem (SHIME (R)). Environ Microbiol Rep, 2013. 5(4): p. 595-603.
18. Aguirre, M., C. Bussolo de Souza, and K. Venema, The Gut Microbiota from Lean and Obese Subjects Contribute Differently to the Fermentation of Arabinogalactan and Inulin. PLoS One, 2016. 11(7): p. e0159236.
19. Bodera, P., Influence of prebiotics on the human immune system (GALT). Recent Pat Inflamm Allergy Drug Discov, 2008. 2(2): p. 149-53.
20. Bock, S. A., et al., Double-blind, placebo-controlled food challenge (DBPCFC) as an office procedure: a manual. J Allergy Clin Immunol, 1988. 82(6): p. 986-97.
21. Brand, P. L. and M. A. Landzaat-Berghuizen, Differences between observers in interpreting double-blind placebo-controlled food challenges: a randomized trial. Pediatr Allergy Immunol, 2014. 25(8): p. 755-9.

1. A method of ameliorating one or more symptoms of an allergy in a subject, comprising:
a) identifying a subject suffering from an allergy;
b) administering a probiotic to the subject; and
c) exposing the subject to an allergen that is causative of said allergy, in an amount sufficient to illicit an immune response, but not an anaphylactic response.

2. The method of claim 1, wherein said probiotic is selected from the group consisting of: a) Clostridium Butyricum; b) Lactobacillus Rhamnosus and; c) Bifidobacterium Longum.

3. The method of claim 1, wherein subject is administered a prebiotic before administration of the probiotic.

4. The method of claim 3, wherein said prebiotic is selected from the group consisting of: a) inulin; b) vitamin D3; and c) an oligosaccharide.

5. The method of claim 1, wherein said allergen is derived from a source selected from the group consisting of: a) peanuts; b) wheat; c) soy; d) fish; e) shellfish; f) tree nuts; g) dairy, and h) eggs.

6. The method of claim 1, wherein said allergen is derived from a source selected from the group consisting of: a) animal dander, b) pollen, c) grass, d) mold, e) insect stings, f) dust mites, g) latex, and h) animal saliva.

7. The method of claim 1, wherein said allergy occurs after or as a result of microbiome dysbiosis in the gut.

8. The method of claim 7, wherein said allergy occurs after or as a result of exposure to one or more antibiotics.

9. The method of claim 1, wherein exposing said allergen comprises internally administering the allergen to the subject.

10. The method of claim 1, wherein exposing said allergen comprises external exposure of the allergen to the subject.

11. The method of claim 1, wherein the probiotic and the allergen are administered contemporaneously or within 2 hours of each other.

12. The method of claim 1, wherein the subject is administered probiotics comprising: Lactobacillus Rhamnosus and Bifidobacterium Longum.

13. The method of claim 12, wherein the probiotics are administered at between 1×10⁸ and 1×10¹² CFUs.

14. The method of claim 12, wherein the subject is further administered Bifidobacterium infantis.

15. The method of claim 14, wherein the subject is further administered Bifidobacterium Acidophilus.

16. The method of claim 12, wherein the probiotics are administered for at least 7 days.

17. The method of claim 1, wherein exposing the allergen to the subject comprises an allergen immunotherapy protocol.
